(19) United States
(12) Patent Application Publication
(10) Pub. No.: US 2003/0190704 A1
(43) Pub. Date: Oct. 9, 2003

(54) Methods and compositions for anchoring stem cells in a microenvironment

(76) Inventor: Ting Xie, Leawood, KS (US)

Correspondence Address: POLSINELLI SHALTON & WELTE, P.C., 700 W. 47TH STREET, SUITE 1000, KANSAS CITY, MO, 64112-1802, US

(21) Appl. No.: 10/174,677

(22) Filed: Jun. 19, 2002

Related U.S. Application Data
(60) Provisional application No. 60/370,114, filed on Apr. 4, 2002.

(51) Int. Cl. C12P 21/02; C12N 5/06; C12N 5/08

(52) U.S. Cl. 435/69.1; 435/325; 435/348; 435/366

(57) ABSTRACT

The present invention relates to various glycoproteins, such as cadherins and related nucleic acid molecules, which are used to anchor and attract stem cells to a specified niche. Location of the stem cells in the niche can result in cell differentiation and the regeneration of surrounding tissue.

The present application is a non-provisional application, which is based on Provisional Patent Application Serial No. 60/370,114, filed Apr. 4, 2002.

### FIELD OF THE INVENTION

The present invention relates to methods and compositions for anchoring stem cells in various microenvironment niches, whereby the stem cells can undergo lineage commitment into differentiated mature tissue cells. The present invention includes the use of various nucleic acid molecules and amino acid sequences for anchoring and, more specifically, attracting and holding the stem cells, in particular, adult somatic or germline stem cells.

### BACKGROUND OF THE INVENTION

The theory that stem cells can be used to regenerate damaged or dead tissue cells is well known. Knowledge of stem cell biology is crucial for the development and study of therapies used to treat a wide range of diseases. Because stem cells play essential roles ranging from embryonic development and organogenesis (fetal stem cells, including embryonic stem cells) to tissue homeostasis and regeneration (adult stem cells), it is hypothesized that stem cells could be used, for example, to repair nerve cells or replace damaged skin cells. Related to this, it is desired to use adult stem cells to repair developed'1 tissue. Assuming adult stem cells can be used, converting non-differentiated adult stem cells into selected differentiated mature tissue cells is difficult for a variety of reasons. Consequently, it is desired to have methods and model systems, which utilize and allow for the study of adult stem cells.

Stem cells exist in many adult tissues and evolve into differentiated mature cells that replace cells lost during a host animal's lifetime. Understanding the molecular mechanisms controlling stem cell function in vivo is crucial to the future use of stem cells in regenerative medicine, as well as for understanding aging, tumor formation, and degenerative diseases. Recently, it has been shown that adult stem cells have great “plasticity” in terms of producing differentiated progeny. What is not completely understood is how or what causes a stem cell to remain as a stem cell and differentiate. For this reason, it is desired to have a method, composition, and model system for determining how a stem cell self-renews and differentiates. It is more desired to have a method, composition, and model system, which can be used to induce selected stem cell differentiation and lineage commitment. This is especially desired because by controlling commitment, methods for regeneration of adult tissue cells can likely be practiced.

Stem cells are defined by the ability to self-renew and generate cell populations that differentiate to maintain adult tissues. Stem cell behavior is thought to be controlled by neighboring stromal cells, such as cap cells, that create special microenvironments known as stem cell “niches” whose regulatory potential persists even when stem cells are absent.

One of the priorities in stem cell research is to further define structures and functions of different stem cell niches and reveal the molecular mechanisms involved in the communication between stem cells and their niches. It is believed that such communications influence stem cell differentiation. The niches relate to areas where stem cells are attached in a microenvironment. In particular, a niche is an area where the stem cell is adhered to and contacts other cells, whereby signaling will occur, which maintains the stem cell and directs cell differentiation and lineage commitment. This is known as the “niche” hypothesis. Specifically, the “niche” hypothesis postulates that stem cells reside in optimal microenvironments or niches. When a stem cell divides, only one daughter can remain in the niche, while the other becomes committed to differentiate. A stem cell within the niche has a high probability of self-renewal but a low probability of entry into the differentiation pathway. This model is consistent with the observations that stem cells require the addition of growth factors for proliferation and differentiation in many in vitro culture systems.

The molecular nature of the microenvironment within a niche, it is believed has yet to be defined in any system; however, the Drosophila germarium appears to contain such a niche (FIG. 1a). To understand the niche, especially the microenvironment, the structure and genes expressed should be defined. Anteriorly in the germarium, the stem cells abut terminal filament and cap cells which both express hedgehog (hh), while only the latter express wingless (wg) and armadillo (arm) (Forbes et al., 1996a, 1996b). The genes, decapentaplegic (dpp), fs(1)Yb(Yb), piwi, wg, arm, and hh are known to be important for germline stem cell (GSC) maintenance. Stem cell daughters lie more to the posterior and probably directly contact inner germarial sheath cells, which express hh and patched (Forbes et al., 1996b). This asymmetry in structure and signals may allow germline stem cells to receive different levels of signals from their daughters. Consistent with the existence of a niche, it is believed that a vacated niche can be reoccupied by stem cells. From the above and known references, it is concluded that a model for studying stem cell adherence exists.

An exemplary system for studying stem cells in a niche are germline stem cells in a Drosophila germarium. In the germarium, the GSCs will differentiate into nurse cells and oocytes. The germarium is the interior portion of the ovariole in an insect, with the ovariole forming part of the ovary. It is in the germarium where cystocyte division occurs (generation of nurse cell/oocyte clones), and clusters of cystocytes become enveloped by follicle cells. The GSCs produce cystoblasts at the anterior tip of the germarium. Each cystoblast gives rise to one oocyte and 15 nurse cells, which are enclosed by an epithelial sheet of somatic follicle cells, thus forming an egg chamber. The egg chamber consists of 16 interconnected cystocytes surrounded by a monolayer of follicle cells, with the oocyte the most posterior cystocyte. In insects, there are three types of ovaries, panoistic and meroistic, which includes polytrophic meroistic ovaries and telotrophic meroistic ovaries. In the meroistic ovaries, both oocytes and nurse cells are generated. In a telotrophic meroistic ovary, the nurse cells are restricted to the germarium and are connected to oocytes in early stages of their development by cystoplasmic processes called nutritive chords.

In each egg chamber, the oocyte is always positioned at the posterior end, whereas the 15 nurse cells are located at the anterior end. The germ cells, which are connected by intercellular bridges, called ring canals, are closely associated with one another and with the follicle cells. As the cytoplasmic materials of nurse cells flow into the oocyte through these ring canals, the oocyte grows in size. At stage 9 of oogenesis, the follicle cell epithelium retracts from the nurse cells and subsequently a subset of follicle cells, called the centripetal follicle cells, grows inward along the nurse cell—oocyte border. At about the same time as the follicle cell retraction, a small number of another type of follicle cell—the border cells—segregate from the follicle cell epithelium at the anterior end of the egg chamber and migrate between the nurse cells to the anterior end of the oocyte. From the above, it is concluded that Drosophila oogenesis includes a variety of morphogenetic events, which are useful for studying cell commitment and differentiation. The above is further useful to provide an overview of a model system for use in studying stem cell adherence and commitment.

Once the GSCs are anchored in the germarium, they are typically in contact with cap cells. The cap cells are believed to signal the GSCs and cause them to remain as stem cells. As such, it is important for the GSC daughters to be in close proximity to inner sheath cells so that differentiation into an oocyte can occur. From the above information, the following questions emerge. What anchors and attracts the GSCs to the germarium and, in particular, to contact the cap cells? Does the same system impact adult somatic stem cells?

GSCs in the adult Drosophila ovary provide an acceptable adult stem cell system in which stem cells and niches can be studied in vivo at both the cellular and molecular levels. At the tip of the germarium (see FIG. 1a), which is located at the beginning of each ovariole of an ovary, a niche exists for two to three GSCs whose progeny eventually develop into mature oocytes. GSCs can be reliably identified based on their size, location, and the shape of their fusome (a germ cell-specific intracellular structure rich in membrane skeleton proteins). Stem cells usually contain a round fusome but exhibit a distinctively elongated shape, while transiently connected to their daughter cells following division. In addition, the stem cells in the germarium directly contact cap cells and are close to two other somatic cell types, terminal filament cells and inner germarial sheath cells (see FIG. 1a). As mentioned, terminal filament and cap cells express the genes, dpp, fs(1)Yb, piwi, and hh that are known to be important for GSC maintenance. In order to understand how niches might regulate GSCs, it is necessary to understand how GSCs are maintained in their niches. For this reason, it is desired to have a method or composition for maintaining a GSC, or related adult stem cell, in a microenvironment in vivo.

A genetic analysis of wg and arm in the ovarian system previously indicated that the wg-arm signaling cascade is not required for the differentiation and development of the germ-line in the ovary (Baker 1988; Peifer 1995). This indicates that something else impacts cell differentiation.

It is known that, for example, the shg or shotgun (SEQ. ID NO. 110) gene expresses an E-cadherin protein (SEQ. ID NO. 3). In previous publications, it has been hypothesized that the shg/E-cadherin system is required for close association between germ cells in the egg chamber (insect ovarium follicle) and cell migration. In particular, it was hypothesized that the shg gene contributes to organized cell adhesion, and that the shg mediates oocyte positioning. It was further hypothesized that the shg gene worked in conjunction with the arm gene to form adherens junctions in the egg chamber. E-cadherin-mediated cell adhesion has been shown to play an important role in the interactions between germ cells and follicle cells during Drosophila oogenesis. This adhesion requires two essential components, E-cadherin and β-catenin, that are encoded by the shg and arm genes in Drosophila, respectively. It has not been known, however, that the E-cadherin protein is required for the GSCs to attach to cap cells. In particular, it has not been known that cadherins are required for cell anchorage to cells other than proximal GSCs.

Cell-to-Cell adhesion is important for the development of multicellular organization. Intercellular junctions usually develop at the cell-to-cell contact sites between neighboring cells, and several types of junctions, including tight junctions (TJs), adherens junctions (AJs) and desmosomes, occur in epithelial cells. These junctions form junctional complexes at the most apical part of the lateral membrane.

Adherens junctions are composed of a cadherin—catenin complex and its associated proteins. Recently, an increasing number of novel members of adherens junctions, including membrane and PDZ proteins, have been reported. Interactions among these components in adherens junctions seem to be dynamically regulated during the formation of adherens junction complexes in epithelial cells.

While all of the above demonstrate that GSC and other stem cells can be held together, it is yet to be determined what attracts and holds a stem cell in a niche. Methods for attracting and anchoring adult stem cells in an environment have not been known. It is known that in certain microenvironments or niches, the stem cells are contacted by other cells, and cell differentiation and commitment occur. The differentiation of the adult stem cells into a specific cell type is believed to be primarily determined by the niche. It is further known that if a stem cell or group of stem cells are placed in a niche, they often diffuse away from the niche without the desired commitment. It is desired to have methods and compositions for holding the stem cell in a niche until commitment occurs. It is even more desired to have a method for regeneration of tissue cells, using adult stem cells.

It is believed that methods and compositions for anchoring stem cells within a germarium are not known. It is further believed that methods for anchoring germ cells in a microenvironment are not known. It is desired to have a method and composition for anchoring a stem cell in vivo. It is especially desired to have a method for anchoring an adult stem cell in a desired niche. It is finally desired to have a model system and method for studying how lineage commitment occurs in GSCs.

### SUMMARY OF THE INVENTION

The present invention relates to a method for anchoring and attracting stem cells, in particular, adult stem cells, in vivo to a microenvironment, or niche. The niche is formed from adult tissue cells, which communicate with and cause stem cell commitment. As such, a particular niche will cause a stem cell to differentiate into a particular adult tissue cell. Importantly, the present method can be used to anchor a stem cell in a selected niche, whereby the stem cell can differentiate into a selected tissue cell to regenerate or replace damaged or dead tissue cells. The method can also be used to maintain a stem cell in a niche. The method will include using either a nucleic acid molecule or an amino acid sequence to cause attraction and attachment of the stem cell to the selected niche.

The present method includes isolating a gene or nucleic acid molecule which, when expressed, produces an adhesive amino acid sequence or protein, preferably a glycoprotein. The protein is then contacted with a selected microenvironment, thereby causing a stem cell or cells to attach to the microenvironment. It is known that the stem cells will communicate with the cells, which form the microenvironment or niche, whereby lineage commitment in the stem cells will occur. In turn, adult tissue cells can be regenerated. Additionally, the stem cells can be maintained in the niche.

A method for anchoring stem cells in vivo in a microenvironment is initiated by isolating a stem cell and expressing a nucleic acid molecule in the stem cell to produce an adhesive protein, such as the glycoprotein. The stem cell can then be placed in a selected micronenvironment and attached thereto. The stem cells will communicate with cells, which form the microenvironment, whereby lineage commitment in the stem cells will occur. The gene for use in an isolated stem cell should express a glycoprotein, preferably, a cadherin.

The stem cells can originate from any member of the Animalia kingdom, including insects and mammals. The stem cells derived from mammals include human, pig, goat, dog, rat, mouse, and a variety of other mammals. Somatic or germline stem cells may be used, as well as adult or embryonic stem cells. As such, any of a variety of stem cells can be used, as long as they can be adhered to a microenvironment, and differentiation into a tissue cell occurs. It is preferred to use adult germline or somatic stem cells.

The microenvironment can be comprised of any of a variety of cells that cause lineage commitment in the stem cell that contacts the niche. Also, the niche can be formed in any of a variety of tissues. Suitable tissues include nerve, skin, or kidneys, for example. The selected tissues will be those where it is desired to have cell regeneration. The microenvironment or niche is typically formed by stromal cells, such as cap cells. As such, the resultant differentiated stem cell can be used as part of a regeneration or homeostatis process in various tissues. The niche is derived from a Drosophila, a non-human mammal, or a human.

Suitable nucleic acid molecules include an shg or arm gene, including SEQ. ID NOs. 110-117, combinations of the genes, and complementary sequences thereof. Degenerate variants of the sequences of nucleic acid molecules and genes are also available for use. A preferred isolated nucleic acid molecule is one that encodes a cadherin protein, especially an E-cadherin. Isolated nucleic acid molecules, which are at least 50% homologous to the mentioned nucleic acid molecules, may be used. Additionally, molecules, which are at least 60%, 75%, or 90% homologous to the nucleic acid molecules are also useful. Any nucleic acid molecule that expresses a cadherin, which can be used to anchor a stem cell in a niche, may, however, be used.

Once a nucleic acid molecule is isolated an expression vector can be formed, which causes stem cells to be anchored in a niche. The vector will include a promoter operably linked to any of the mentioned nucleic acid molecules. Optionally, a marker may also be included. The expression vector should result in the cell expressing a protein to cause adhesion and attachment in the microenvironment. A viral vector capable of directing expression of the nucleic acid molecule is an example of a suitable expression vector. The vector can then be used to transfect a host cell. The host cell can be of a non-human mammalian, human, or insect origin. As before, it is preferred for the host cell to be a stem cell. An isolated oligonucleotide that binds to the nucleic acid molecule can be derived from the nucleic acid molecules.

Expression can be controlled by injection of an shg, arm, or combination thereof encoding nucleic acid molecule into a niche. Expression can also be controlled by delivery of an shg or arm nucleic acid molecule by micro-vessels.

A method for producing a protein that causes stem cells to be anchored in a niche is available. The method includes culturing a host cell that contains a vector, under conditions and for a time sufficient to produce the protein. The stem cell will be placed in the selected niche.

Any of a variety of proteins or amino acid sequences may be used, as long as cells can be adhered and, preferably, attracted to the niche. The adhesive protein is preferably a glycoprotein and, more preferably, a cadherin protein, such as E-cadherin. Any adhesive protein can be used that will attract and attach stem cells to a niche. A wide variety of cadherins can be used that cause adherence. Selection of a specific cadherin will typically be determined by the species and cell type. The available proteins include an isolated protein comprising SEQ. ID NOs. 1-109. E-cadherin and β-catenin proteins encoded by the nucleic acid molecules previously mentioned can be used in combination. This is preferred. Isolated proteins that are 90% homologous thereto may also be used. The proteins should be amino acid sequence, which cause adhesion of the stem cell in a niche

Antibodies, which specifically bind to the proteins may be used, as well as hybridomas that express the antibodies. Such antibodies are desired for testing and identification. Probes for isolating proteins that cause stem cell anchoring are developed herewith, whereby the probes are comprised of one of the previously mentioned proteins. Probes that are at least 90% homologous are also available. Additionally, cDNA probes formed from the mentioned isolated nucleic acids are contemplated, as are antibodies that bind specifically to an shg or arm protein. An antibody that selectively binds to an epitope in the receptor-binding domain of the shg or arm protein can be used.

Methods for purifying a glycoprotein or cadherin protein from a biological sample containing glycoprotein or cadherin protein are practiced. The method includes providing an affinity matrix comprising an antibody bound to a solid support, followed by contacting the biological sample with the affinity matrix, to produce an affinity matrix-cadherin protein complex separation, separating the affinity matrix-cadherin protein complex from the remainder of the biological sample and releasing the cadherin protein from the affinity matrix.

A kit for detecting an shg gene or cadherin protein may also be formed. Either kit will have a container and a nucleic acid or amino acid molecule. This suggests that specific regulatory microenvironments or “niches” must exist that can reprogram one stem cell type to another in order to produce various progeny in different tissues.

It has been determined that E-cadherin-mediated cell adhesion is essential for recruiting stem cells to niches during niche formation and anchoring the stem cells, especially GSCs, in their niche in the adult Drosophila ovary. E-cadherin belongs to a family of transmembrane adhesion molecules that are expressed in a variety of tissues and cell types. In a similar manner, E-cadherin or related adhesion molecules (such as DE-cadherin) may also help recruit and anchor stem cells in their niches in other organisms, including humans. Increasing the expression of E-cadherin or related adhesion molecules on the surface of stem cells will allow stem cells to find their niches and remain there, thus allowing them to maintain their stem cell phenotype. Studies provide a basis for the identification and studying of E-cadherin and/or related adhesion molecules to determine their role in recruiting and holding stem cells in their niches. This will, most likely, assist future efforts to use stem cells in regenerative medicine.

### BRIEF DESCRIPTION OF THE DRAWINGS

The application file contains at least one photograph executed in color. Copies of this patent application publication with color photographs will be provided by the Office upon request and payment of the necessary fee.

FIG. 1a is an explanatory diagram of a Drosophila germarium in cross section indicating germline stem cells (GSCs, yellow) cystoblasts (CBs, white), developing cysts (DCs, white), spectrosomes (SS, dark green), fusomes (FS, dark green), terminal filament cells (TFs, light pink), cap cells (CPCs, blue), inner germarial sheath cells (IGS, brown), somatic stem cells (SSCs, light green) and follicle cells (FCs, light green);

FIG. 1b is a micrograph of an anterior half of a wild-type germaria, labeled with anti-E-cadherin and anti-Hts antibodies, whereby a GSC in contact with a cap cell is shown;

FIG. 1c is a micrograph of an anterior half of a wild-type germaria, labeled with anti-E-cadherin and anti-Hts antibodies, whereby a GSC with an elongated fusome in contact with a cap cell is shown;

FIG. 1d is a micrograph of an anterior half of a wild-type germaria, labeled with anti-E-cadherin, anti-Hts antibodies, and DAPI, cap cells, cap cell GSC junctions, and GSC nuclei are shown;

FIG. 1e is a micrograph of an anterior half of a wild-type germaria, labeled with anti-E-cadherin, anti-Hts antibodies, and DAPI, cap cells, cap cell GSC junctions, and GSC nuclei are shown;

FIG. 1f is a micrograph of an anterior half of a wild-type germaria, labeled with anti-E-cadherin, anti-Hts antibodies and DAPI, cap cells, cap cell GSC junctions, and GSC nuclei are shown;

FIG. 1g is a micrograph of an anterior half of a wild-type germaria, labeled with anti-E-cadherin, anti-Hts antibodies, and DAPI, showing co-localization of E-cadherin and arm;

FIG. 2a is a schematic showing generation of marked shg mutant GSC clones;

FIG. 2b is a photomicrograph of one-week old germaria, labeled with anti-LacZ and anti-E-cadherin, E-cadherin accumulation between wild-type GSCs and cap cells is shown, as is accumulation between mutants and cap cells;

FIG. 2c is a photomicrograph of one-week old germaria, labeled with anti-LacZ and anti-E-cadherin, E-cadherin accumulation between wild-type GSCs and cap cells is shown, as is accumulation between mutants and cap cells;

FIG. 3a is a timeline graph illustrating how long wild-type and mutant GSCs are carried in a germaria as a function of time;

FIG. 3b shows a germarium marked with anti-LacZ, anti-Hts, and DAPI, carrying a mutant cyst generated by a lost mutant shg^{R69} GSC two weeks after clone induction;

FIG. 3c shows a germarium marked with anti-LacZ, anti-Hts, and DAPI, carrying three wild-type unmarked GSCs with a mutant shg^{R69} cyst in a late egg chamber of the same ovariole;

FIG. 3d shows a germarium marked with anti-LacZ, anti-Hts, and DAPI, carrying three marked shg¹⁰⁴⁶⁹ GSCs three weeks after clone induction;

FIG. 3e shows a germarium marked with anti-LacZ, anti-Hts, and DAPI, carrying three-week old marked wild-type GSC clone;

FIG. 3f shows a germarium marked with anti-LacZ, anti-Hts, and DAPI, carrying three-week old arm² GSC clone;

FIG. 3g shows a germarium marked with anti-LacZ, anti-Hts, and DAPI, carrying three-week old double arm⁸ GSC clones;

FIG. 3h shows a germarium marked with anti-LacZ, anti-Hts, and DAPI, carrying two unmarked GSCs with a marked arm⁴ GSC differentiating into a cyst two weeks after clone induction;

FIG. 3i shows a germarium marked with anti-LacZ, anti-Hts, and DAPI, carrying a wild-type GSC and a mutant GSC (not shown on this confocal section but producing marked cysts), demonstrating that the wild-type GSC has expanded its contact with multiple cap cells;

FIG. 3j shows one of the few and rare three-week-old double arm⁴ GSC clones, which may have derived from loss of wild-type GSCs followed by replacement with a mutant one;

FIG. 4a is a photomicrograph of late third instar larval ovaries immunostained with anti-E-cadherin (red) and anti-Hts (green) antibodies;

FIG. 4b is a photomicrograph of the third instar larval ovary showing no expression of E- cadherin on juxtaposing membranes (indicated by black arrows) between GSCs and cap cells;

FIG. 4c is a photomicrograph showing two ovarioles showing punctate patterns of E-cadherin expression on the membranes (indicated by black arrows) between GSCs and cap cells;

FIG. 4d is a photomicrograph of an ovariole showing E-cadherin expression at a high level on the membranes (indicated by black arrows) between GSCs and cap cells (an overlay of multiple confocal sections);

FIG. 4e is a photomicrograph of a germarium carrying three marked wild-type GSC clones; and,

FIG. 4f is the current model for anchorage of GSCs in their niches, red bars between GSCs and cap cells indicate the presence of E-cadherin and Arm on contacting membranes between GSCs and cap cells, most likely in the form of adherens junctions.

### DETAILED DESCRIPTION

The present invention relates to methods and compositions for anchoring stem cells in a niche or microenvironment, where signaling occurs, which initiates lineage commitment and causes a stem cell to differentiate into an adult cell. The differentiated adult stem cells can be used in tissue homeostasis and regeneration. In particular, the niche influences the particular type of differentiation so that if the stem cell can be anchored in a pre-determined niche, the resultant adult cell can not only be predicted, but essentially chosen. For example, an undifferentiated stem cell can be anchored in a niche defined by nerve cells, whereby the differentiated nerve cells will cause lineage commitment in the stem cell and, ultimately, differentiation of the stem cell into a nerve cell. The present invention can also be used to maintain a stem cell in a niche and promote self-renewal. As such, the invention relates to at least one nucleic acid molecule, and at least one protein or amino acid sequence, which cause the stem cell to be attracted and adhered to a chosen niche. In particular, the present invention relates to the use of adhesive proteins or amino acid sequences to attract and hold the stem cells. Families of amino acid sequences used to anchor stem cells are part of the present invention. The present method can be used as part of a tissue regeneration method.

Specifically, the invention relates to and includes isolated shg nucleic acid molecules and related genes and nucleic acid molecules. Also included are proteins and amino acid sequences expressed therefrom, in particular, cadherins. Any of a variety of amino acid sequences, especially cadherins, can be used herewith. As such, nucleic acid molecules or genes, which can be expressed to form such proteins, are available for use. Expression vectors, which are formed from the available nucleic acid molecules may be used herewith, as well as host cells, which have been transfected with the vectors. Isolated oligonucleotides that bind to one of the nucleic acid molecules are used herewith. Antibodies, which specifically bind to the proteins or amino acid sequences, and probes for isolating the proteins are further part of the present invention.

Yet another part of the present invention relates to methods for causing adherence of the stem cell in a microenvironment or niche. The present invention also relates to nucleic acid molecules and amino acid sequences, which can be used as part of a kit or method to detect suitable adhesive compositions, especially suitable glycoproteins. Finally, the present invention relates to methods for purifying the cadherin proteins and kits for detecting the shg gene and related shg alleles, as well as the related nucleic acid molecules and proteins.

A stem cell is one of the mitotically active somatic cells that serve to replenish those that die during the life of a metazoan organism, as well as one of the mitotically active germinal cells that produce a continuing supply of gametes. Stem cells are cells that, while not themselves critical parts of muscle or blood, grow into cells that make such tissues. Thus, they are the ‘stem’ from which the ‘leaves’ of tissue arise. Stem cells, therefore, have two roles: to make more stem cells, and to make differentiated ‘progeny’ cells. A somatic cell is any cell of the eukaryotic body other than those destined to become germline or sex cells. Either germline or somatic stem cells may be used in the present invention. It is preferred to use the somatic cells as part of a method for repairing or regenerating tissue.

As used herein, stem cells can be derived from a variety of host organisms, including any derived from the kingdom Animalia. The stem cells can be derived from insects and mammals, including both human and non-human mammals. Suitable non-human mammals will include dogs, cats, mice, rats, goats, and livestock. In particular, the stem cells can be either adult or embryonic; however, it is preferred to use adult stem cells. As mentioned, the adult stem cells include both germline stem cells (GSCs) and somatic stem cells.

The stem cells are maintained in “niches” or specific regulatory microenvironments formed by stromal cells, which includes cap cells. How stem cells are recruited to and maintained in their niche is crucial to understanding stem cell regulation and for using stem cells in regenerative medicine. As stated, it is important for stem cells to be held in a particular microenvironment or niche. This will cause specific lineage commitment or differentiation in the stem cell and cause the stem cell to mature into a differentiated cell that forms tissue. Available tissues that can likely be repaired using the present method include skin, nerve, muscle, kidney, pancreatic, occular, and a variety of other tissue systems. At issue is a population of cells of the same kind, performing the same function. The niche is an area the stem cell can be attracted to and held. Once in position, the stem cell will receive signals from the surrounding cells which form the microenvironment. These biochemical signals will cause lineage commitment in the stem cell. The stem cells can also be anchored in the testis or ovary to cause proliferation of GSCs. As such, the present method can be used to regenerate tissue in a variety of insects and mammals, including humans, mice, dogs, cats, and livestock.

An isolated stem cell includes a nucleic acid molecule, which can be over-expressed and is selected from the group of isolated nucleic acid molecules comprising SEQ. ID NOs. 110-117, or complementary sequences thereof, degenerate variants of the sequences of the listed nucleic acid sequences, and any isolated nucleic acid molecule that encodes a cadherin protein and can be used herewith.

Any of a variety of adhesive molecules can likely be used to anchor the stem cell in a selected microenvironment. The adhesive molecule can be derived from the same genus or species to be treated, or from a different genus or species. An adhesive amino acid sequence molecule, typically a protein, that can attract and attach stem cells to a microenvironment niche where differentiation can be initiated, may be used (define “adhesive molecules”). A preferred adhesive protein is a glycoprotein, which is a protein containing small amounts of carbohydrates. Suitable glycoproteins include members of the cadherin family. Cadherins are glycoproteins composed of between 700 and 750 amino acids that function as cell-cell adhesion molecules. The N-terminal of the molecule projects from the membrane surface and contains Ca²⁺ binding sites. The C-terminal tail binds to the actin of the cytoskeleton. In between is a segment that functions as an integral part of the cell membrane.

Cadherins play an important role in specific cell-cell adhesion events. Their expression appears to be tightly regulated during development and each tissue or cell type shows a characteristic pattern of cadherin molecules. The cadherins form a superfamily with at least six subfamilies, which can be distinguished on the basis of protein domain composition, genomic structure, and phylogenetic analysis of the protein sequences. These subfamilies comprise classical or type-I cadherins, atypical or type-II cadherins, desmocollins, desmogleins, protocadherins and Flamingo cadherins.

E-cadherin is a transmembrane glycoprotein with an extracellular part comprising five ectodomain modules (EC) of about 110 amino acid residues with conserved calcium binding sequence motifs. As such, E-cadherin has four homologous cadherin repeats (EC1 to EC4) and one less related membrane-proximal extracellular domain (EC5 or MPED). The complete ectodomain is indeed stabilized by multiple calcium ions. The repeats EC1 to EC4 are typical of the various cadherin molecules, and range in number from four (classical cadherins) to 34 (FAT). Through these repeats, many of the different cadherins mediate cell-cell adhesion. Each cadherin repeat was found to fold into a seven-stranded β-barrel structure. Interdomain rigidification is achieved by the binding of three calcium ions to highly conserved peptide sequences (LDRE, DXNDN and DXD). Most, (if not all) other cadherins are expressed specifically in various organs.

E-cadherins are the best characterized of the cadherins. They are known to be present in many types of epithelial cells and are usually concentrated in the adhesion belts that hold the cells together. Drosophila E-cadherin is equally related to E and N-cadherin in mammals. Cadherin is the most preferred glycoprotein for use herewith. Examples of other suitable cadherins include: N-cadherin, P-cadherin, R-cadherin, VE-cadherin, K-cadherin, cadherin 7, cadherin 8, T1-cadherin, T2-cadherin, OB-cadherin, N-cadherin 2, N-cadherin 2, H-cadherin, M-cadherin, KSP-cadherin, LI cadherin, cadherin 18, cadherin 19, cadherin 11, and cadherin 20. More specifically, available cadherins are as follows:

| | | | | Extracellular |
|---|---|---|---|---|
| | Synonyms (or names of | Gene | Species | cadherin |
| Protein name | ortholognes) | symbol | (gene location) | domains |
| BS-cadherin | | BS-CDH | Bs | 5 |
| Cadherin-1 | E-cadherin, uvomorulin, L-CAM | CDH1 | Hs (16q22.1 | 5 |
| Cadherin-2 | N-cadherin | CDH2 | Hs (18q12.1 | 5 |
| Cadherin-3 | P-cadherin, B-cadherin^{d}, | CDH3 | Hs (16q22.1) | 5 |
| | XB/U-cadherin^{d} | | | |
| Cadherin-4 | R-cadherin, XmN-cadherin | CDH4 | Hs (20q123.3 | 5 |
| Cadherin-5 | VE-cadherin | CDH5 | Hs (16g22.1) | 5 |
| Cadherin-6 | K-cadherin | CDH6 | Hs (5p14-15.1) | 5 |
| Cadherin-7 | | CDH7 | Hs (18q22-23) | 5 |
| Cadherin-8 | | CDH8 | Hs (16g22.1) | 5 |
| Cadherin-9 | Ti-cadherin | CDH9 | Hs | 5 |
| Cadherin-10 | T2-cadherin | CDH10 | Hs (Sp13-14) | 5 |
| Cadherin-11 | OB-cadherin | CDH11 | Hs (16q22.1) | 5 |
| Cadherin-12 | Br-cadherin, N-cadherin-2 | CDH12 | Hs (Sp15.1-15.2) | 5 |
| Cadherin-13 | T- or H-cadherin | CDH13 | Hs (16g24.2) | 5 |
| Cadherin-15 | M-cadherin | CDH15 | Hs (16g24.3) | 5 |
| Cadherin-16 | Ksp-cadherin | CDH16 | Hs (16g21-22) | 7 |
| Cadherin-17 | LI-cadherin, HPT-1 | CDH17 | Hs (8g22.1-22.3) 7 | |
| Cadherin-18 | Cadherin-14, mouse EY-cadherin | CDH18 | Hs (5p15.1-15.2 | 5 |
| Cadherin-19 | | CDH19 | Hs (18q22-23) | 5 |
| Cadherin-20 | F-cadherin^{e}, mouse “cadherin-7”^{e} | CDH20 | Hs (18g22-23) | 5 |
| Cdh-3 | | CE-CDH3 | Ce | 19 |
| Dachsous | | DACUSOUS | Dm | 27 |
| Desmocollin-1 | | DSC1 | Hs (18g12.1) | 5 |
| Desmocollin-2 | | DSC2 | Hs (18q12.1) | 5 |
| Desmocollin-3 | | DSC3 | Hs (18q12.1) | 5 |
| Desmoglein-1 | | DSG1 | Hs (18g12.1) | 4 |
| Desmoglein-2 | | DSG2 | Hs (18g12.1) | 5 |
| Desmoglein-3 | | DSG3 | Hs (18q12.1) | 5 |
| DE-cadherin | | DE-CDH | Dm | 6 |
| DN-cadherin | | DN-CDH | Dm | 15 |
| EP-cadherin | C-cadherin | EP-CDU | X1 | 5 |
| Fat | | FAT | Hs (4q34) | 34 |
| Flamingo | Starry night (Stan) | FM1 | Dm | 9 |
| Flamingo 1 | | FMI1 | Mm | 8 |
| HMR1 | | HMR1 | Ce | 2 |
| MEGF1 | Fat2 | MEFG1 | Rn | 34 |
| MEGF2 | Celsr1 | MEGF2 | Rn | 8 |
| Paraxial | 6 | | | |
| protocadherin | PAPC | PAR-PCDH | X1 | |
| pB-cadherin | | PB-CDH | Rn | 5 |
| Protocadherin-α1 | CNR family member | PCDH-α | Hs (5g31-33 | 6 |
| Protocadherin-β1 | Protocadherin-3 family member | PCDH-β1 | Hs (5q31-33 | 6 |
| Protocadherin-β15 | Protocadherin-2 family member^{f} | PCDH-β15 | Hs (5q31-33 | 6 |
| Protocadherin-γa1 | Protocadherin-2 family member^{f} | PCDH-γ1 | Hs (5q31-33) 6 | |
| Protocadherin-γb1 | Protocadherin-2 family member^{f} | PCDH-γB1 | Hs (5q31-33) | 6 |
| Protocadherin-γc3 | Protocadherin-2 family member^{f} | PCDH-γC3 | Hs (5q31-33) | 6 |
| Protocadherin-1 | Protocadherin-42 | PCDH1 | Hs (5q31-33) | 7 |
| Protocadherin-4 | VE-cadherin-2 | PCDH4 | Mm | 6 |
| Protocadherin-7 | BH-Protocadherin,NF-protocadherin | PCDH7 | Hs (4p15) | |
| Protocadherin-8 | Arcadlin | PCDH8 | Hs (13q21) | 6 |
| Protocadherin-10 | OL-Protocadherin | PCDH10 | Mm | 6 |
| Protocadherin-11 | Protocadherin-X | PCDH11 | Hs (Xq21.3/Yp11) | 7 |
| Protocadherin-68 | | PCDH68 | Hs | 6 |
| Ret | | RET | Hs | 7 |

In the above table, Hs stands for Homo sapien; DM stands for Drosophila melanogaster; X1 stands for Xenopus laevis; Mm stands for Mus musculus; Ce stands for Caenorhabditis elegans; Rn stands for Rattus norvegicus; and Bs stands for Botryllus schlosseri.

SEQ. ID NO. 1 is a β-catenin. The cadherins that can be used herewith are SEQ ID NOs. 2-109. This includes Drosophila and Homo sapien cadherins. In particular, SEQ. ID NOs. 2-6 relate to specific cadherins derived from a Drosophila species. SEQ ID NOs. 7-109 relate to Homo sapien proteins. SEQ ID NO. 110 relates to an shg gene derived from Drosophila. The arm gene of SEQ ID NO. 111 is derived from Drosophila. SEQ ID NO. 112 relates to Drosophila N-cadherin gene. SEQ ID NO. 113 is a Drosophila E-cadherin-1 gene. SEQ ID NO.114 is a Homo sapien Cadherin 5 gene. SEQ ID NO.115 is a Homo sapien Cadherin-3, P-cadherin gene. SEQ ID NO. 116 is a Homo sapien, E-cadherin gene. SEQ ID NO. 117 is a Homo sapien cadherin-4, R-cadherin gene.

The cadherin selected for use with the present invention will depend upon the species and the selected niche. Cadherins can also be used in combination with other adhesive proteins, such as β-catenin. More particularly, a fragment of the ≢-catenin will be used. The selected fragment will promote attachment. Use of the fragment is preferred because β-catenin can impact Wnt signaling and resultingly cause development problems. Thus, only the attachment portion of the β-catenin will be used.

As shown in the Examples, E-cadherin-mediated cell adhesion is important in anchoring germline stem cells (GSCs) in their niches in the Drosophila ovary. E-cadherin and β-catenin have been observed to be the major components of this adhesion process. E-cadherin and β-catenin are encoded by shg and arm, respectively. These proteins were observed to accumulate in high levels in the junctions between GSCs and cap cells, one of the niche components. Removal of these proteins from GSCs resulted in stem cell loss. Furthermore, E-cadherin is also required to recruit GSCs to their niches. This demonstrates that anchorage of GSCs in their niches by E-cadherin-mediated adhesion is important for stem cell maintenance and function.

Any nucleic acid molecule or gene which expresses a protein or amino acid sequence which can be used to attract and anchor stem cells in a niche may be used herewith. Preferably, the gene or nucleic acid molecule will express a glycoprotein, in particular, a cadherin. The preferred gene for use herewith is an shg, which express E-cadherin. As stated, the shg gene naturally occurs in insects, specifically Drosophila, and mammals, including humans. Available genes or nucleic acid molecules are listed in Table 1.

The Drosophila shg gene is identified as SEQ. ID NO. 110. Genes or nucleic acid sequences, which function in the same way, may be used herewith. Genes from species other than Drosophila, which are in the same family and express an adhesive amino acid sequence, may also be used.

Complementary sequences to the listed shg nucleic acid molecules may be used with the present invention. As would be expected, a complementary sequence is one that can be expressed to form a protein that causes attraction and adherence. Further, degenerate variants of the sequences may be used. Degenerate variants are those in which each different word is coded by a variety of symbols or groups of letters. The present degenerate variants include at least one nucleotide triplet that is different but codes for the same amino acid.

Nucleic acid molecules homologous to the shg nucleic acid molecules may be used to attract and attach the stem cells. The homologous nucleic acid molecules can be identified using a NCBI BLAST Program. Suitable homology will include those nucleic acid molecules that are 50% homologous to the listed shg gene and related genes. More preferably, the homology will be 60% and, even more preferred, 75% homologous to the shg gene. The most preferred homologous nucleic acid molecule will be 90% homologous to the shg gene. Homologous nucleic acid molecules may be derived from humans, non-human mammals, and insects.

Mutant alleles of the shg nucleic acid molecule may be used as part of a kit or a particular method. The mutants can be used for testing purposes.

Isolated oligonucleotides can be derived from the nucleic acid molecules. The oligonucleotides are the active portions of the molecules, and cause attraction and adherence of the stem cells. Such oligonucleotides can be used with the present invention.

Expression vectors, which cause attraction and attachment of the stem cells, can be formed from the above-discussed nucleic acid molecules, using known procedures. A promoter will be operably linked to the isolated nucleic acid molecule, such as the shg gene, to form the expression vector. Any promoter which causes expression of the nucleic acid molecule, and can be turned on and off, can be used. Suitable promoters can be selected from the group consisting of tubilin, actin, and any of a variety of cadherin promoters. It is further preferred to include a marker with the vector, such as ampicillin. Suitable vectors include DNA vectors, plasmid vectors, and shuttle vectors. It is preferred to form a vector having an shg gene, which can be turned on to cause over-expression of the shg gene.

Once a vector is formed, it can be used to transfect a host cell, whereby a transgenic host cell will be produced that incorporates the vector that expresses the nucleic acid molecule, which causes anchorage and attraction of the stem cell. In particular, the vector can be used to transfect a stem cell and cause over-expression of the cadherin. Methods for transfecting the host cell are well known, and comprise culturing the vectors with the host cells. The host cell can be of any of a variety of origins, including mammalian- or insect-derived cells. More preferably, the host cells are derived from non-human mammals and humans.

A transgenic animal can be formed using the present invention. In particular, transgenic non-human mammals and insects can be formed, which include the mentioned nucleic acid molecules.

The proteins or amino acid sequences expressed by the shg gene and related nucleic acid molecules, as mentioned, should cause attraction to and attachment in a niche and can be isolated and purified. Such isolation and purification include known procedures and methods, including affinity chromotography or purification. The isolated proteins or amino acid sequences include those listed herein as SEQ. ID NOs. 2-109. Additional, suitable proteins or amino acid sequences, include those encoded by an shg nucleic acid molecule, and proteins, which are 90% homologous with the protein of SEQ. ID NOs. 2-109. As would be expected, proteins that are 50% homologous to the protein of SEQ. ID NOs. 2-109 may also be used, with proteins 60% homologous more preferred. A protein that is 75% homologous to SEQ. ID NOs. 2-109 is even more preferred. Homology of the proteins is determined according to the BLAST program. As such, any of a variety of proteins may be used, as long as they are expressed by an shg gene or homologous nucleic acid molecule, and cause extraction and attachment to a microenvironment.

Proteins expressed from mutants of shg may also have other uses with the present invention. Such mutant proteins may be used for testing purposes.

Probes used to isolate the above proteins and/or genes, can be formed from the proteins or genes. The probes include cDNA, mRNA, and monoclonal and polyclonal antibodies. All the probes are formed using known procedures. Probes, which are 50% homologous to the proteins, may also be formed. More preferably, the probes will be 75% and, even more preferably, 90% homologous to the above proteins. Homology of these probes is determined according to the BLAST program. Antibodies, which specifically bind to the above-listed proteins, are part of the present invention. Additionally, hybridomas that produce such antibodies may be used herewith.

In addition to protein probes, cDNA probes may be formed, which are comprised of isolated nucleic acid molecules as listed above. As such, any antibody, which binds specifically to an shg or arm nucleic acid molecule, may be used. Antibodies that selectively bind to an epitope in the receptor-binding domain of the shg or arm protein may also be used.

The isolated proteins may be used as part of a method for anchoring stem cells in a niche. Once the shg gene or other discussed nucleic acid molecule is expressed to form the protein or amino acid sequence, such protein can be isolated and purified. The isolated and purified protein or amino acid sequence is then contacted with, or placed in a niche with stem cells, whereby the stem cells will be held in place. The niche may be derived from any of a variety of species, including insects and mammals. More preferably, the niche is derived from a Drosophila species or a human species. Non-human mammals may be treated with the isolated protein. The protein is placed in contact with the niche by way of any of a variety of different methods. As such, any method that allows the protein to contact the niche and attract and attach a stem cell is suitable.

The present method will also include over-expressing the shg gene or related nucleic acid molecule in the stem cell to cause adherence. In this method, stem cells can be removed from a host organism. A vector having the shg gene can transfect the stem cell. The gene will be activated to over-express the protein. The stem cell is then reinserted into the host in vivo in a selected niche, where it is desired to have additional cells. This can be part of a tissue regeneration method.

Most cells may be transfected using a vector. Other known methods can be used to transfect a host cell and express the desired gene. For example, a calcium phosphate system may be used. An injection process may be used, whereby a nucleic acid molecule is injected into the niche, and the molecule is then expressed to produce the desired protein. Microinjection processes are known and can be practiced with the present technology. An alternative method involves delivery via a micro-vessel.

As would be expected, methods for purifying the shg or cadherin protein from a biological sample containing such proteins are important. A preferred method includes providing an affinity matrix with the antibody to cadherin bound to a solid support. A biological sample is contacted with the affinity matrix, to produce an affinity matrix-shg protein complex. Next, the affinity matrix-shg protein complex is separated from the remainder of the biological sample, with the shg protein released from the affinity matrix.

In the present method, the number of stem cells placed in vivo in a niche will be dependent upon the size or space available in the niche. Typically, between 1 and 10 stem cells will be placed in a niche. The preferred amount of adhesive amino acid sequence will be greater than what is normally expressed.

A kit for detecting an shg gene can be formed. The kit will preferably have a container and a nucleic acid molecule, which includes SEQ. ID NOs. 110-117.

A kit for detecting an shg protein can also be formed. The kit will preferably have a container and a nucleic acid molecule, which includes SEQ. ID NOs. 2-109.

The arm gene and protein are useful for use in combination with shg and cadherin. Attachment and attraction of the stem cells can be enhanced when the arm and shg are used in combination. Any nucleic acid molecule which expresses β-catenin protein or a related amino acid sequence, which can be used to attract and anchor stem cells in a niche, may be used herewith. Preferably, the gene or nucleic acid molecule will express a β-catenin or a related molecule. As stated, the arm gene naturally occurs in insects, specifically Drosophila, and mammals, including humans. The arm gene in Drosophila is identified as SEQ. ID NO. 111, and the protein expressed therefrom (β-catenin) is identified as SEQ. ID NO. 1.

The proteins expressed by the arm gene and related nucleic acid molecules can cause attachment in a niche and can be isolated and purified. Such isolation and purification include known procedures and methods, including affinity chromatography or purification. The isolated proteins or amino acid sequences include SEQ. ID NO. 1. Additional, suitable proteins or amino acid sequences include those encoded by an shg nucleic acid molecule, and proteins, which are 90% homologous with the proteins of SEQ. ID NOs. 2-109. As would be expected, proteins that are 50% homologous to the proteins of SEQ. ID NOs. 2-109 may also be used, with proteins 60% homologous more preferred. A protein that is 75% homologous to SEQ. ID NOs. 2-109 is even more preferred. Homology is determined according to the BLAST program. As such, any of a variety of proteins may be used, as long as they are expressed by an shg gene or homologous nucleic acid molecule, and cause extraction and attachment to a microenvironment.

A kit for detecting an arm gene can be formed. The kit will preferably have a container and a nucleic acid molecule, which includes SEQ. ID NO. 111.

A kit for detecting a β-catenin protein can also be formed. The kit will preferably have a container and a nucleic acid molecule, which includes SEQ. ID NO. 1.

The following examples are for illustrative purposes only and are not to be construed as limiting the scope of the subject invention.

### EXAMPLES

### Example 1.

To determine whether E-cadherin-mediated cell adhesion is important for anchoring GSCs in their niche, or microenvironment, the expression of E-cadherin and arm protein in the germarium was examined. E-cadherin and arm were observed to be present in the junctions formed between GSCs and cap cells.

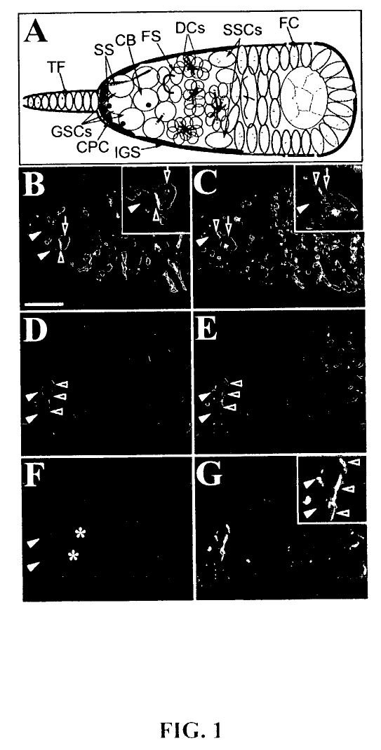

The study was initiated by taking micrographs (confocal sections) to show the anterior halves of wild-type germaria after immuno-fluorescent labeling with anti-E-cadherin (red) and anti-Hu-li Tai Shao (anti-Hts, green) antibodies, shown in FIGS. 1b and 1c. Hts protein is a molecular marker for the fusome. This was done to ensure that a wild-type system was available for comparison. Labeling of the germaria was done according to known procedures. Also, the micrographs were made according to known procedures. As can be seen, only one GSC with a round fusome (insert B, indicated by an arrow) or an elongated fusome (insert C, indicated by an arrow), indicating two interconnected stem cell daughters, was shown to contact cap cells (indicated by white arrowheads). As will be discussed, the micrograph revealed a number of structures in the germarium.

E-cadherin proteins were observed to accumulate in high levels at the contact sites between GSCs and CPCs (indicated by black arrowheads, FIGS. 1b and 1c). This demonstrates accumulation of the proteins at these sites. Thus, the wild-type showed the presence of GSCs and E-cadherin, proximal to one another.

### Example 2.

The same germarium of Example 1 was further stained. The germarium is shown at FIGS. 1d, 1e and 1f and was immuno-stained with anti-E-cadherin (red), anti-arm (green) and DAPI (DNA, blue), respectively. The germaria were also immuno-stained for Hts protein. The staining process was done according to known procedures. White arrowheads, black arrowheads and *s indicate cap cells, CPC-GSC junctions, and GSC nuclei, respectively. As can be seen, the β-catenin, and E-cadherin were present, with the GSCs located nearby.

The co-localization of E-cadherin and arm are shown at FIG. 1g. This demonstrated that β-catenin and E-cadherin work together. All the micrographs discussed in the present Example and Example 1 are shown at the same scale and the bar in (B) represents 10 μm.

The photos show that E-cadherin and arm proteins accumulated in the contact sites between cap cells and GSCs in high levels, and between cap cells, see FIGS. 1b-1e. Arm and E-cadherin co-localized in these sites, as shown in FIGS. 1d-1g. The distribution of these proteins was not uniform in the contact areas between GSCs and cap cells. Instead, it was observed that there were focal sites between the cells that were rich in E-cadherin and arm proteins. These foci formed at areas adjacent to the fusome, FIGS. 1b and 1c. Since E-cadherin-based cell adhesion in long-term cell-cell contacts often forms adherens junctions, it was hypothesized that these foci are adherens junctions. Thus, it was determined that E-cadherin-mediated cell adhesion between cap cells and GSCs was involved in anchoring GSCs to cap cells. Most importantly, it was shown that E-cadherin collects, or is localized, in the germarium. This was an important in proving that E-cadherin can be used to hold GSCs in place.

### Example 3.

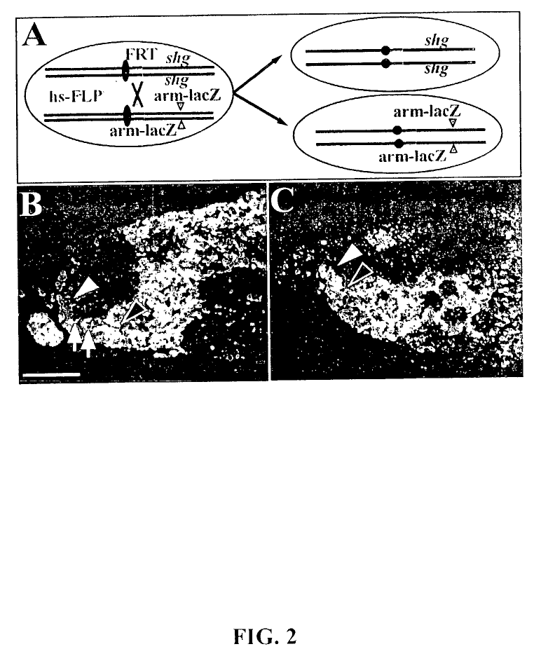

To investigate whether E-cadherin-mediated cell adhesion is essential for maintaining stem cells in their niche, marked GSCs that lack functional copies of either shg or arm, were produced using FLP-mediated FRT recombination. Thus, to investigate whether the removal of E-cadherin from GSCs can disrupt E-cadherin accumulation in the contact sites between cap cells and GSCs, a deletion allele, shg^{R69} was prepared. The marked GSC cells were identified by their loss of expression of an arm-LacZ marker (the fusion of the arm promoter and the bacterial LacZ gene). FIG. 2a is a schematic showing how the marked GSC cells were produced.

The mutant resulted in the removal of E-cadherin proteins from GSCs in the adult ovary. It was observed that the removal of E-cadherin proteins from GSCs diminished the accumulation of E-cadherin in the junctions between GSCs and cap cells.

Germanium bearing shg mutant stem cell clones were generated according to published experimental procedures (10). All cells (ovals) expressed the arm-LacZ marker (green) except shg mutant clones generated by FRT-mediated recombination in the diagram. A cystoblast requires 4 to 5 days to exit the germarium. Therefore, the only remaining LacZ cells one week after clone induction consisted of mutant stem cells and/or their progeny (10). Furthermore, the position of a LacZ mutant cyst in the germarium indicates when its marked GSC was lost.

One week-old germaria in FIGS. 2b and 2c were labeled with anti-LacZ (green) and anti-E-cadherin (red). The accumulation of E-cadherin proteins between a mutant shg^{R69} GSC and cap cells was observed to be greatly reduced (FIG. 2b, white arrowhead) or completely diminished (FIG. 2c, white arrowhead). Such observation is clearly shown in the photos by the reduction in red pigment. Levels remained normal between a wild-type GSC and cap cells, and among cap cells (indicated by white arrows) in the same germaria (B and C, black arrowheads). All micrographs are shown at the same scale and the bar in (B) represents 10 μm.

It was concluded that the removal of shg from GSCs diminished E-cadherin accumulation in the junctions between GSCs and cap cells within one week (FIGS. 2b-2c). Partial depletion was observed in some cases, which may be due to the perdurance of the protein (FIG. 2b). In the germaria containing shg^{R69} GSCs, the accumulation of E-cadherin was still maintained between wild-type GSCs and cap cells. In addition, the removal of E-cadherin proteins from GSCs also diminished the accumulation of arm in the junctions between cap cells and GSCs. This further demonstrated that GSCs migrated out of the germarium prematurely, if E-cadherin was not present in sufficient quantities.

### Example 4.

The present Example was designed to determine whether loss of the shg function resulted in cells not adhering, or migrating from the germarium. Again, it is believed that E-cadherin and arm are required for anchoring GSCs in their niches. This Example focused on proving this hypothesis.

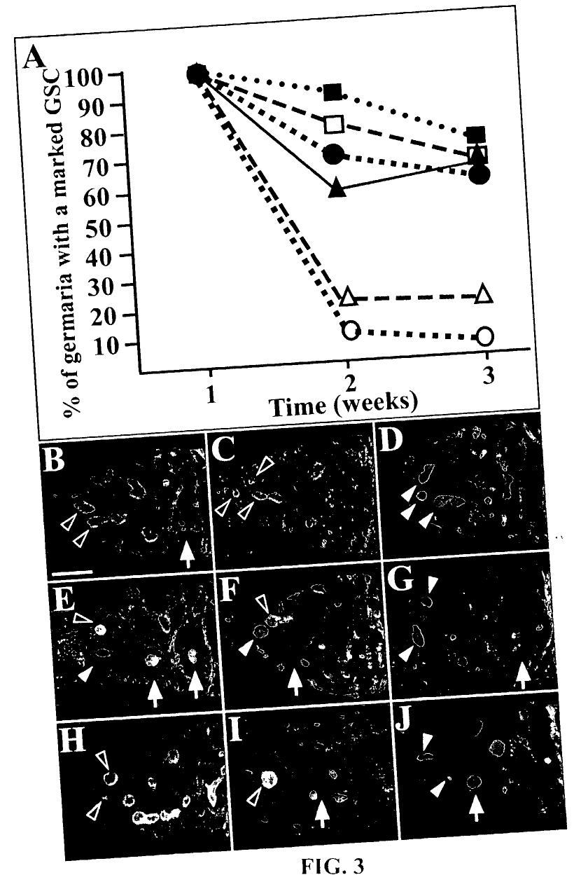

To further determine the importance of E-cadherin-mediated adhesion in maintaining GSCs, two shg alleles, the deletion allele (shg^{R69}) of Example 3 and a weak allele, shg¹⁰⁴⁶⁹ were formed. Mutant shg^{R69} GSCs migrated from the germarium in a comparatively short period of time after they were produced. Due to this quick loss, marked shg^{R69} GSCs occurred at a much lower frequency in germaria in the first week following clone induction (10.0%, n=251), compared to control (n=32.1%, n=109) and shg¹⁰⁴⁶⁹ (n=26.8%, n=149) germaria. This is shown in the graph of FIG. 3a. FIG. 3a shows the percentage of germaria carrying marked wild-type or mutant GSCs as a function of time. The various cells were marked as follows: Wild-type (▪), shg¹⁰⁴⁶⁹ (●), shg^{R69} (◯), arm² (□), arm⁴ (Δ) and arm⁸ (▴). Since individual experiments started with different percentages of germaria carrying marked GSCs, the results were normalized to 100% for comparison at the first week. The number of germaria examined for each mutant at every time point ranged from 149 to 360.

The data shown in FIG. 3a is supported by the photo-micrographs of FIGS. 3b-3j. Germaria samples (b-j) were labeled with anti-LacZ (red), anti-Hts (green) and DAPI (DNA, blue). White arrows indicate marked GSCs. Fusomes of unmarked GSCs are denoted by black arrowheads, while marked cysts are shown by arrows.

During the first two weeks, germaria carrying one or a few shg^{R69} mutant cysts without marked GSCs were observed (FIG. 3b), suggesting recently lost mutant GSCs. In addition, more than 95% of marked shg^{R69} GSCs observed during the first week were lost within two weeks (FIGS. 3a, 3c). The weak allele, shg¹⁰⁴⁶⁹ had no dramatic effect on GSC maintenance, with 60% of marked GSCs from the first week being maintained for two more weeks (FIG. 3a). In addition, germaria carrying one, two, or three of these marked GSCs were frequently observed three weeks after clone induction (FIG. 3d). In germaria carrying two or three marked GSCs, one or two marked GSCs had apparently replaced one or two wild-type GSCs in the germarium as determined by the presence of wild-type cysts in late egg chambers. As a control, 70% of marked wild-type GSCs observed one week after clone induction were maintained in their niches for two more weeks (FIGS. 3a, 3e). From this, it was concluded that the mutant clonal analysis results demonstrate that E-cadherin is essential for anchoring GSCs in their niche.

### Example 5.

Like Example 4, GSC adherence was analyzed. The photos of FIG. 3 are part of the present Example. Here, instead of analyzing E-cadherin, β-catenin adherence was analyzed. Two classes of arm mutants were used to differentiate the role of arm in GSC adhesion, since arm is involved in wingless (wg) signaling and E-cadherin-mediated adhesion. The arm² and arm8 alleles were used for their ability to specifically block wg signaling while having no obvious effects on cell adhesion, while arm⁴ was chosen for its defects in both wg signaling and cell adhesion. About 60% of marked GSCs mutant for arm² and arm⁸ observed in the first week following their induction were maintained for two more weeks, suggesting that disrupting wg signaling did not dramatically effect the GSC maintenance (FIGS. 3a, 3f, 3g). In contrast, arm had severe effects on GSC maintenance. A lower percentage of arm⁴ mutant GSC clones were observed during the first week (1.5%, n=200) after clone induction, due to rapid mutant GSC loss, in comparison with those for arm² (9.4%, n=159) and arm⁸ (6.8%, n=219). Furthermore, over 80% of the marked arm⁴ GSCs were lost three weeks after clone induction (FIGS. 3a, 3h).

To further exclude the possibility that disrupting wg signaling contributes to GSC loss, marked GSCs mutant for disheveled and shaggy (two essential downstream components in wg signaling) were generated. These mutant GSCs were maintained normally in their niche, further indicating that wg signaling does not play a direct role in GSC maintenance. It was concluded from the above results that arm may be required for anchoring GSCs in their niche through cadherin-mediated cell adhesion.

### Example 6.

Using the data and materials of Example 4, the following observations were made. In view of Examples 4 and 5, one of the possible explanations for GSC loss caused by shg and arm mutations is that mutant GSCs cannot compete with wild-type GSCs for contact with cap cells, since they are defective in cell adhesion. Consistent with this interpretation, a wild-type GSC in a germarium carrying a mutant GSC sometimes expanded its contact area with cap cells, as shown in FIG. 3i. In addition, in the absence of wild-type GSCs in a germarium (all GSCs were mutated for either arm⁴ or shg^{R69}), marked mutant GSCs were persistent even three weeks after clone induction, as shown in FIG. 3j. This suggested that the identity of mutant GSCs can be maintained, as long as they remain in their niche and are not required to compete with wild-type GSCs.

### Example 7.

E-cadherin-mediated cell adhesion is involved in intracellular signaling in many different systems. It has been previously shown that signaling from surrounding somatic cells is important for GSC division. To study whether E-cadherin-mediated adhesion is important for GSC division, the relative division rates for shg mutant GSCs were measured. (Give protocol) The relative division rate (rdr) for a given genotype was calculated by dividing the number of marked cysts per marked GSC by the number of unmarked cysts per unmarked GSC in a given germarium. In comparison with marked wild-type GSCs (rdr=1.05, n=132), shg¹⁰⁴⁶⁹ had no obvious effect on GSC division (rdr=0.98, n=161), while shg^{R69} had a weak effect on GSC division (rdr=0.82, n=26). These results indicate that disrupting E-cadherin-mediated cell adhesion appears to have only a weak effect on GSC division. It is concluded that cadherin does not cause cell commitment.

### Example 8.

To determine the relationship between E-cadherin and the development of GSCs, the expression of E-cadherin in late third instar larval ovaries was examined. E-cadherin-mediated cell adhesion is required for recruiting GSCs to niches during niche formation.

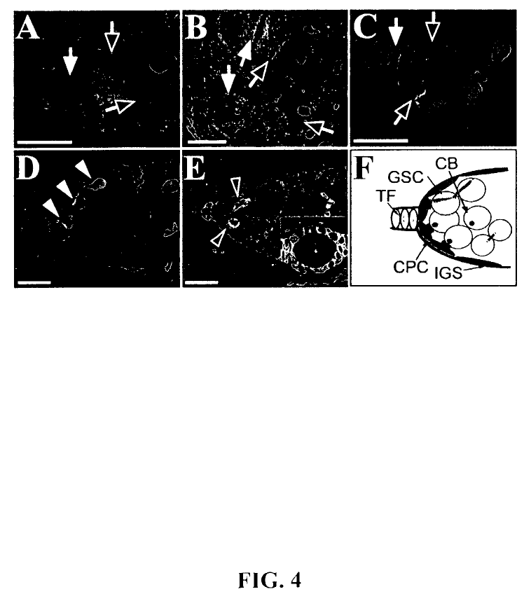

Late third instar larval ovaries, as shown in FIGS. 4a-4c, were immuno-stained with anti-E-cadherin (red) and anti-Hts (green) antibodies. E-cadherin was expressed in 8-10 fully developed terminal filament cells (indicated by white arrows) and developing cap cells. An ovariole showing no expression of E-cadherin on juxtaposing membranes (indicated by black arrows) between GSCs and cap cells is shown in FIG. 4a. At this stage, 8-10 terminal filament cells were fully developed, and cap cells were starting to form. This suggested that niche development was still occurring. The patterns of E-cadherin expression in different ovarioles were not completely synchronous within the ovaries.

The following additional observations were made. Two ovarioles showed punctate patterns of E-cadherin expression on the membranes (indicated by black arrows) between GSCs and cap cells, FIG. 4b. Conversely, an ovariole showing E-cadherin expression at a high level on the membranes (indicated by black arrows) between GSCs and cap cells (an overlay of multiple confocal sections) was observed, FIG. 4c.

In all ovarioles, E-cadherin was present between terminal filament cells and the GSCs, as shown in FIGS. 4a-4c. However, E-cadherin expression between cap cells and GSCs was variable, and its relative abundance appeared to depend upon the location of the future GSC's fusome. When the fusomes of future GSCs were not localized to anterior contact sites, E-cadherin expression was either absent (FIG. 4a) or punctate (FIG. 4b). However, in ovarioles, in which fusomes of future GSCs have been localized to the anterior sites where stem cells contact cap cells, E-cadherin accumulated in high levels between cap cells and GSCs (FIG. 4c). It is hypothesized that after the cap cells differentiate, E-cadherin in the cap cells and GSCs initially helps keep both cell types in juxtaposition. Ultimately, it was concluded that permanent contact sites are established where E-cadherin and arm accumulate in high levels and may form adherens junctions.

### Example 9.

To further determine if E-cadherin-mediated cell adhesion is required for recruiting GSCs to their niche, wild-type, mutant shg¹⁰⁴⁶⁹, and shg^{R69} primordial germ cells (PGCs) were marked just before the late third instar stage using the FLP-mediated FRT recombination technique. The efficiency of these marked PGCs to be recruited to their niches in 1-2 day-old females was analyzed.

Germaria, FIGS. 4d and 4e, from 1-2 day old females were labeled with anti-LacZ (red), anti-Hts (green) and DAPI (DNA, blue). All the fusomes of marked GSCs are indicated by white arrowheads, while those of unmarked GSCs are denoted by black arrowheads. Females developed from late third-instar larvae in which some of the PGCs were genetically marked by the loss of arm-LacZ gene with or without a mutant for shg^{R69}.

A germarium carrying three marked wild-type GSC clones (indicated by white arrowheads) is shown in FIG. 4d. A germarium carrying two wild-type unmarked GSCs (indicated by black arrowheads) with mutant shg^{R69} germline cells in egg chambers in the same ovariole is shown in FIG. 4e. The insert shows a mutant stage 2 chamber in the same ovariole.

Only a fraction of PGCs were incorporated into niches and became GSCs. However, if E-cadherin-mediated cell adhesion is important for initial interactions between cap cells and GSCs, it is expected that marked wild-type and mutant shg PGCs would have different efficiencies of incorporation into niches. Marked wild-type PGCs (control) were incorporated into 17.3% of niches (n=294) (FIG. 4d), similar to the marked shg¹⁰⁴⁶⁹ mutant PGCs (13.1% incorporation, n=236). However, PGCs homozygous for the deletion allele, shg^{R69}, were only incorporated into 0.4% of niches (n=252) with the majority of them directly differentiating into germline cysts within egg chambers (FIG. 4e). Therefore, disrupting the expression of E-cadherin in PGCs prevented them from being recruited to their niche and, thus, from becoming GSCs.

The above Examples suggest a working model explaining how GSCs are recruited to and anchored in their niches. The proposed model is shown at FIG. 4f. Red bars between GSCs and cap cells indicate the present of E-cadherin and arm on contacting membranes between GSCs and cap cells, most likely in the form of adherens junctions. All bars represent 10 μm. It is known that E-cadherin proteins accumulate on the contacting membranes between cap cells and GSCs from the beginning of niche establishment, which is important for recruiting GSCs to niches, explaining the intimate relationship observed between cap cells and GSCs. In addition, it has also been demonstrated that E-cadherin and arm are essential for holding GSCs against cap cells and preventing them from moving away from their niche and, therefore, from differentiation. Since terminal filament cells and cap cells express genes that are known for their role in maintaining GSCs, it is suggested that one function mediated by the E-cadherin-based cell adhesion is to ensure that GSCs stay within the niche (in close proximity to cap cells) and, thus, maintain their stem cell identity.

### Example 10.

To study the behavior of cells in the ovary when DE-Cadherin activity is completely removed, germ-line clones homozygous for shg^{1H}, were generated utilizing the dominant female-sterile insertion, P[ovo^{D1}], on the second chromosome. Crosses were made to produce larvae heterozygous for shg^{1H} and ovo^{D1} (shg^{1H}/ovo^{D1}). These were then subjected to X-ray irradiation during larval development to induce mitotic recombination. The recombination generated two distinct genotypes of cell clones, one homozygous for shg^{1H} (shg^{1H}/Shg^{1H}), and the other homozygous for ovo^{D1}(ovo^{D1}/ovo^{D1}). Oogenesis only continued in ovaries that contained shg^{1H}/shg^{1H} germline cells.

Using monoclonal antibodies recognizing the extra-cellular domain of the molecule essential for cell adhesion (Oda et al. 1994a), DE-cadherin was not detected on the surfaces of germ cells homozygous for shg^{1H}. Instead, the translated products were, abnormally accumulated in the cytoplasm (FIG. 3), suggesting an absence of functional adhesion molecules. These results are consistent with the notion that the shg^{1H} mutation is null (Tepass et al. 1996; Uemura et al. 1996).

### Example 11.

The mutants of Example 10 were again analyzed. The cellular morphology of shg^{1H} mutant egg chambers was examined by DIC optics, and by florescence-phalloidin staining to visualize F-actin. It was observed that every mutant egg chamber contained the correct number of germ cells, one oocyte and 15 nurse cells, in their appropriate positions (FIGS. 4c, 4i). The nurse cells and oocyte in shg^{1H} mutants were normally connected to each other by ring canals (FIGS. 3d, 3j). Despite such a normal appearance of the overall morphology of the egg chamber, all the germ cells showed a significant alteration in cell shape. They were rounded, and had lost their tight association with one another and with the follicle cell layer (FIGS. 3c, 3i). Phalloidin staining revealed the presence of intercellular gaps between germ cells at various corners (FIG. 4d). In wild-type egg chambers, germ cells were tightly packed without any intercellular spaces (FIGS. 3a, b, g, h). These results suggest that the mutant germ cells were not normally adherent to each other, nor to the follicle epithelium, consistent with the expected roles for DE-cadherin in cell-cell adhesion. The above findings provide genetic evidence that there is a cadherin-dependent cell migration process. Mutants of arm also showed an occasional failure in the correct migration of border cells (Peifer et al 1993). (Expand)

### Example 12.

A method can be practiced, whereby a protein or amino acid sequence can be used to anchor a stem cell in a niche in vivo. The method is initiated by isolating an shg gene. The gene is then expressed to produce an E-cadherin protein. The E-cadherin protein is then placed in a desired niche, whereby it will be used to help attract and anchor stem cells to the niche.

### Example 13.

A method for using a nucleic acid molecule to cause an adult stem cell to be attached to a microenvironment. The method is initiated by isolating an shg gene. A host stem cell is isolated and transfected with the nucleic acid molecule, shg gene. A vector is used to transfect the host cell. The host stem cell is then placed in a niche, and the gene is expressed to produce an E-cadherin protein. This will be used to help attract and anchor stem cells to the niche.

### Example 14.

In the Drosophila testis, GSCs contact and are regulated by neighboring somatic cells, hub cells that are proposed to function as a niche for GSCs. It is observed that similar junctions also exist between GSCs and hub cells. These results suggest that E-cadherin-mediated cell adhesion may play a similar role in the Drosophila testis. (Expand)

Thus, there has been shown and described a novel method and composition for adhering and attracting stem cells to a microenvironment niche, which fulfills all the objects and advantages sought therefore. It is apparent to those skilled in the art, however, that many changes, variations, modifications, and other uses and applications for the subject method and composition are possible, and also such changes, variations, modifications, and other uses and applications which do not depart from the spirit and scope of the invention are deemed to be covered by the invention, which is limited only by the claims which follow.

What is claimed is:

1. A method for anchoring a stem cell in a niche, comprising:
(a) isolating a nucleic acid molecule which, when expressed, produces an adhesive protein;
(b) expressing the nucleic acid molecule to produce the protein; and,
(c) contacting the protein with a selected niche of cells thereby causing the stem cell to attach to the niche, the stem cell will communicate with the cells which form the niche, whereby lineage commitment in the stem cell can occur, or maintenance of the stem cell will occur.

2. The method of claim 1, wherein the stem cells are derived from the group consisting of insects and non-human mammals.

3. The method of claim 1, wherein the stem cells are human derived

4. The method of claim 1, wherein the stem cells are selected from the group consisting of germline stem cells and somatic stem cells.

5. The method of claim 1, wherein the adhesive protein is a glycoprotein.

6. The method of claim 5, wherein the glycoprotein is selected from the group consisting of E-cadherin, N-cadherin, P-cadherin, R-cadherin, VE-cadherin, K-cadherin, cadherin 7, cadherin 8, T1-cadherin, T2-cadherin, OB-cadherin, N-cadherin 2, N-cadherin 2, H-cadherin, M-cadherin, KSP-cadherin, LI cadherin, cadherin 18, cadherin 19, cadherin 20, and combinations thereof.

7. The method of claim 1, wherein the nucleic acid molecule is selected from the group consisting of SEQ. ID NOs. 110-117.

8. The method of claim 1, wherein the protein is selected from the group consisting of SEQ. ID NOs. 2-109.

9. The method of claim 1, wherein the niche comprises cap cells.

10. The method of claim 1, wherein the nucleic acid molecule is an shg gene.

11. The method of claim 1, wherein the niche comprises cells which cause lineage commitment in the stem cell.

12. The method of claim 1, wherein the nucleic acid molecule is selected from the gene group consisting of shg, arm, and combinations thereof.

13. The method of claim 1, wherein the method is in vivo.

14. The method of claim 1, wherein the niche is formed in tissue selected from the group consisting of ovary and testis that is from Drosophila.

15. The method of claim 1, wherein the niche is formed in a testis that is from a non-human mammal.

16. A differentiated cell formed according to the method of claim 1.

17. A method for anchoring stem cells in a niche, comprising:
(a) isolating at least one adult stem cell;
(b) isolating a nucleic acid molecule which expresses an adhesive protein;
(c) transfecting the stem cell with the nucleic acid molecule;
(d) placing the stem cell in a selected niche of cells; and,
(e) expressing the nucleic acid molecule to produce the adhesive protein, thereby causing the stem cell to attach to the niche, the stem cell will communicate with the cells which form the niche, whereby lineage commitment in the stem cell will occur, or maintenance of the stem cell will occur.

18. The method of claim 17, wherein the niche is located in a selected tissue, and the tissue is selected from the group consisting of nerve, epithelial, kidney, muscle, liver, heart, occular, and pancreatic tissue.

19. The method of claim 17, wherein the nucleic acid molecule is selected from the gene group consisting of shg, arm, and combinations thereof.

20. The method of claim 17, wherein the nucleic acid molecule is selected from the group consisting of SEQ. ID NOs. 110-117.

21. The method of claim 17, wherein the protein is selected from the group consisting of SEQ. ID NOs. 2-109.

22. A method for regenerating tissue in vivo, comprising:
(a) isolating at least one stem cell;
(b) isolating a nucleic acid molecule which expresses an adhesive amino acid sequence;
(c) transfecting the stem cell with the nucleic acid molecule;
(d) placing the stem cell in a niche located in a tissue system where cell regeneration is desired; and,
(e) expressing the nucleic acid molecule to produce the adhesive amino acid sequence, thereby causing the stem cell to attach to the niche, the stem cell will communicate with cells which form the niche, whereby lineage commitment in the stem cell will occur, and the tissue will be regenerated.

23. The method of claim 22, wherein the tissue is selected from the group consisting of nerve, epithelial, kidney, muscle, liver, heart, occular, and pancreatic tissue.

24. An isolated stem cell comprising: a stem cell transfected with a nucleic acid molecule vector, which can be over-expressed, the nucleic acid molecule selected from the group consisting of:
(a) an isolated nucleic acid molecule selected from the group consisting of SEQ.
ID NOs. 110-117, or complementary sequences thereof;
(b) degenerate variants of the sequences of step a; and,
(c) an isolated nucleic acid molecule that encodes a cadherin protein according to (a) or (b).

25. A niche having the isolated stem cell of claim 24.

26. The stem cell of claim 24, wherein the stem cell is selected from the group consisting of non-human mammalian stem cells and insect stem cells.

27. The stem cell of claim 24, wherein the stem cell is selected from the group consisting of somatic and germ stem cells.

28. A method for causing lineage commitment and differentiation in a stem cell, comprising:
(a) isolating at least one adult stem cell;
(b) isolating a nucleic acid molecule which expresses an adhesive protein;
(c) transfecting the stem cell with the nucleic acid molecule;
(d) placing the stem cell in a selected niche; and,
(e) expressing the nucleic acid molecule to produce the adhesive protein, thereby causing the stem cells to attach to the niche, the stem cell will communicate with cells which form the niche, whereby lineage commitment in the stem cell will occur.

29. An isolated nucleic acid molecule which causes stem cells to be anchored in an niche, selected from the group consisting of:
(a) an isolated nucleic acid molecule selected from the group consisting of SEQ. ID NOs. 110- 117, or complementary sequences thereof;
(b) degenerate variants of the sequences of step a; and,
(c) an isolated nucleic acid molecule that encodes an shg protein according to (a) or (b).

30. An isolated nucleic acid molecule comprising a sequence at least 50% homologous to the nucleic acid molecules of claim 29(a).

31. An isolated nucleic acid molecule comprising a sequence at least 60% homologous to the nucleic acid molecules of claim 29(a).

32. An isolated nucleic acid molecule comprising a sequence at least 75% homologous to the nucleic acid molecules of claim 29(a).

33. An isolated nucleic acid molecule comprising a sequence at least 90% homologous to the nucleic acid molecules of claim 29(a).

34. An expression vector, which causes stem cells to be anchored in a niche, comprising a promoter operably linked to a nucleic acid molecule according to any of claims 29 through 33.

35. A method of producing an amino acid sequence that causes stem cells to be anchored in a niche, comprising culturing a cell which contains a vector according to claim 34, under conditions and for a time sufficient to produce the amino acid sequence.

36. A viral vector capable of directing expression of a nucleic acid molecule according to claims 29 through 33.

37. A host cell carrying a vector according to any of claims 34 or 36.

38. An isolated oligonucleotide that binds to the nucleic acid molecule of claim 29.

39. An isolated protein which causes attraction and anchoring of stem cells in vivo in a niche, selected from the group consisting of:
(a) an isolated protein selected from the group consisting of SEQ. ID NO. 2-109;
(b) a protein encoded by the nucleic acid molecules of claim 29;
(c) an isolated protein that is 90% homologous according to step a.

40. An antibody, which specifically binds to the proteins of claim 39.

41. A hybridoma that expresses the antibody of claim 40.

42. A probe for isolating a protein that causes stem cell anchoring, wherein the probe is comprised of the protein of claim 39.

43. The probe of claim 42, wherein the probe is at least 90% homologous to the protein of claim 42.

44. A cDNA probe comprising an isolated nucleic acid consisting of the nucleotide sequence of claim 29.

45. An antibody that binds specifically to a protein expressed by the nucleic acids of claim 22.

46. An antibody that selectively binds to an epitope in the receptor-binding domain of the protein expressed by the nucleic acids of claim 22.

47. A method of purifying a protein from a biological sample containing a protein, comprising:
(a) providing an affinity matrix comprising the antibody of claim 40, bound to a solid support;
(b) contacting the biological sample with the affinity matrix, to produce an affinity matrix protein complex;
(c) separating the affinity matrix protein complex from the remainder of the biological sample; and,
(d) releasing protein from the affinity matrix.

48. A kit for detecting an shg gene, wherein the kit comprises:
(a) a container; and,
(b) a nucleic acid molecule comprising the nucleotide molecules of claim 29.

49. A kit for detecting a cadherin protein, wherein the kit comprises:
(a) a container; and,
(b) a protein of claim 39.

50. A method for anchoring stem cells in a niche comprising, contacting an adhesive protein with a selected niche, thereby causing stem cells to attach to the niche, the stem cells will communicate with cells which form the microenvironment, whereby lineage commitment in the stem cells can occur.

51. An isolated nucleic acid molecule which causes stem cells to be anchored in an niche, selected from the group consisting of:
(a) an isolated nucleic acid molecule comprising SEQ. ID NO. 111, or complementary sequences thereof;
(b) degenerate variants of the sequences of step a; and,
(c) an isolated nucleic acid molecule that encodes an β-catenin protein according to (a) or (b).

52. An isolated nucleic acid molecule comprising a sequence at least 50% homologous to the nucleic acid molecules of claim 51(a).

53. An isolated nucleic acid molecule comprising a sequence at least 60% homologous to the nucleic acid molecules of claim 51(a).

54. An isolated nucleic acid molecule comprising a sequence at least 75% homologous to the nucleic acid molecules of claim 51(a).

55. An isolated nucleic acid molecule comprising a sequence at least 90% homologous to the nucleic acid molecules of claim 51(a).

56. An expression vector, which causes stem cells to be anchored in a niche, comprising a promoter operably linked to a nucleic acid molecule according to any of claims 51-55.

57. A method of producing an amino acid sequence that causes stem cells to be anchored in a niche, comprising culturing a cell which contains a vector according to claim 56, under conditions and for a time sufficient to produce the amino acid sequence.

58. A viral vector capable of directing expression of a nucleic acid molecule according to claims 51 through 55.

59. A host cell carrying a vector according to any of claims 56 or 58.

60. An isolated oligonucleotide that binds to the nucleic acid molecule of claim 51.

61. An isolated protein which causes attraction and anchoring of stem cells in vivo in a niche, selected from the group consisting of:
(a) an isolated protein comprising SEQ. ID NO. 1;
(b) a β-catenin protein encoded by the nucleic acid molecules of claim 51;
(c) an isolated protein that is 90% homologous according to step a.

62. An antibody, which specifically binds to the proteins, according to claim 61.

63. A hybridoma that expresses the antibody of claim 62.

64. A probe for isolating a protein that causes stem cell anchoring, wherein the probe is comprised of the protein of claim 61.

65. The probe of claim 64, wherein the probe is at least 90% homologous to the protein of claim 61.

66. A cDNA probe comprising an isolated nucleic acid consisting of the nucleotide sequence of claim 51.

67. An antibody that binds specifically to arm protein.

68. An antibody that selectively binds to an epitope in the receptor-binding domain of the arm protein.

69. A method for anchoring a stem cell in a niche, comprising:
(a) contacting a protein with a selected niche, thereby causing the stem cell to attach to the niche, the stem cell will communicate with cells which form the niche, whereby lineage commitment in the stem cell can occur; and
(b) the protein is selected from the group consisting of SEQ. ID NOs. 2-6.

70. The method of claim 69, wherein the niche is formed in tissue that is from Drosophila.

71. The method of claim 69, wherein the niche is formed in a testis that is from a non-human mammal.

72. A method for anchoring a stem cell in a niche, comprising:
(a) contacting a the protein with a selected niche, thereby causing the stem cell to attach to the niche, the stem cell will communicate with cells, which form the niche, whereby lineage commitment in the stem cell can occur; and
(b) the protein is selected from the group consisting of SEQ. ID NOs. 2-6.

73. The method of claim 72, wherein the niche is formed in tissue selected from the group consisting of an ovary and testis that is from a non-human mammal.

74. The method of claim 69, wherein the niche is formed in a testis that is from a non-human mammal.

75. A method for anchoring a germline stem cell in a Drosophila niche, comprising:
(a) isolating at least one adult Drosophila germline stem cell;
(b) isolating an shg gene which expresses a cadherin protein;
(c) transfecting the stem cell with the gene;
(d) placing the stem cell in a selected Drosophila GSC niche; and,
(e) expressing the shg gene to produce the cadherin, thereby causing the germline stem cell to attach to the GSC niche, the stem cell will communicate with cells which form the niche, whereby lineage commitment in the stem cell will occur.

76. The method of claim 75, wherein the cadherin is an E-cadherin.

77. The method of claim 75, wherein the protein is selected from the group consisting of SEQ. ID NOs. 2-6.

78. A family of proteins selected from the group consisting of SEQ. ID NOs. 2-6.

79. An antibody which specifically binds to the proteins, according to claim 78.

80. The family of claim 78, wherein the proteins are used in stem cells derived from the group consisting of insects and non-human mammals.

81. The antibody of claim 79, wherein the proteins are used in stem cells, which are human derived.

82. The antibody of claim 79, wherein the proteins are used in stem cells selected from the group consisting of germline stem cells and somatic stem cells.

83. The antibody of claim 79, wherein the proteins are used in a niche, which comprises cap cells.

84. The antibody of claim 83, wherein the niche comprises cells which cause lineage commitment in the stem cell.

85. The method of claim 75, wherein the method is in vivo.

86. The method of claim 1, wherein the niche is formed in tissue selected from the group consisting of an ovary and testis that is from Drosophila.

87. The method of claim 1, wherein the niche is formed in an ovary that is from a non-human mammal.

88. An isolated protein which causes attraction and anchoring of stem cells in vivo in a niche, selected from the group consisting of:
(a) an isolated protein selected from the group consisting of SEQ. ID NOs. 7-109;
(b) a protein encoded by the nucleic acid molecules of claim 29;
(c) an isolated protein that is 90% homologous according to step a.

89. An isolated protein which causes attraction and anchoring of stem cells in vivo in a niche, selected from the group consisting of:
(a) an isolated protein selected from the group consisting of SEQ. ID NOs. 2-6;
(b) a protein encoded by the nucleic acid molecules of claim 29;
(c) an isolated protein that is 90% homologous according to step a.
